(19) United States
(12) Patent Application Publication
(10) Pub. No.: US 2013/0306062 A1
(43) Pub. Date: Nov. 21, 2013

(54) OXYGEN ADMINISTRATION SYSTEM AND METHOD

(71) Applicant: SENSIBLE DISASTER SOLUTIONS, LLC, Bowling Green, OH (US)

(72) Inventor: Jonathan Eric Larson, Bowling Green, OH (US)

(73) Assignee: SENSIBLE DISASTER SOLUTIONS, LLC, Bowling Green, OH (US)

(21) Appl. No.: 13/794,870

(22) Filed: Mar. 12, 2013

Related U.S. Application Data
(60) Provisional application No. 61/649,495, filed on May 21, 2012.

(51) Int. Cl. A62B 7/06; A62B 18/02; A61M 16/06; A62B 9/02

(52) U.S. Cl. CPC A62B 7/06; A62B 9/02; A62B 18/02; A61M 16/0666
USPC 128/201.21; 128/205.24

(57) ABSTRACT

Systems and methods are provided that employ both a liquid oxygen source and a gaseous oxygen source. In this way, a seamless switch between these sources is possible and oxygen can be continuously provided during replacement of a depleted oxygen source. To this end, an oxygen delivery system includes a connection box and dispenser along with the liquid oxygen source and the gaseous oxygen source. The connection box has a common conduit and first, second, and third ports fluidly coupled to the common conduit. The liquid oxygen source is fluidly coupled to the common conduit through the first port and the gaseous oxygen source is fluidly coupled to the common conduit through the second port. The dispenser is fluidly coupled to the common conduit through the third port. Such systems and methods provide uninterrupted oxygen to injured persons and can improve the time and/or effectiveness of recovery.

### CROSS-REFERENCE TO RELATED APPLICATIONS

This application claims the benefit of U.S. Provisional Patent Application Ser. No. 61/649,495, filed on May 21, 2012. The entire disclosure of the above application is incorporated herein by reference.

### FIELD

The present technology relates to oxygen administration systems and methods, including portable systems configured for use by first responders and other healthcare facilities.

### INTRODUCTION

This section provides background information related to the present disclosure which is not necessarily prior art.

Ambient air contains about twenty percent by weight oxygen with the balance being mainly nitrogen. Breathing air with a higher oxygen content has beneficial effects for persons temporarily suffused with certain gases, poisons, fumes, or smoke. For example, persons suffering from smoke inhalation or carbon monoxide poisoning at a disaster site can be promptly treated with oxygen by first responders. Other persons suffering from asthma or other respiratory ailments can likewise be treated with oxygen as a part of first aid. Various athletes, aviators, and high altitude workers can use supplemental oxygen to allay respiratory exertion. Other medical conditions, such as migraines, cardiac disease, and Chronic Obstructive Pulmonary Disease can benefit from treatment with oxygen.

Therapeutic effects of supplementary oxygen include the elimination of nitrogen bubbles in tissue or blood vessels, oxygenation of plasma to increase physically dissolved oxygen, reduction of tissue edema, and increased oxygen saturation of hemoglobin. In each of these examples, the user of a supplementary oxygen delivery system desires to maintain a certain inspired oxygen percentage for a given duration of time. However, in some situations such as in remote locations, the availability of oxygen sources is limited. This makes the efficiency of the delivery system an important factor. The percentage of oxygen required may also differ according to the situation as well. For example, in various emergency applications, as close as possible to 100% inspired oxygen may be desired.

There are issues with delivering adequate quantities of oxygen to one or more persons at a remote location or disaster site. Pressurized oxygen tanks often do not last for a sufficient period while persons are being treated at a site and/or are in transit to a treatment facility, such as a hospital. Portability and the capacity to treat multiple persons are further issues, as first responders may have to physically transport an oxygen delivery system through various obstacles in order to provide treatment to several affected persons.

### SUMMARY

The present technology includes systems, processes, articles of manufacture, and compositions that relate to oxygen administration systems and methods.

In some aspects, an oxygen delivery system is provided that includes a connection box, a liquid oxygen source, a gaseous oxygen source, and a dispenser. The connection box includes a common conduit and first, second, and third ports fluidly coupled to the common conduit. The liquid oxygen source is fluidly coupled to the common conduit through the first port. The gaseous oxygen source is fluidly coupled to the common conduit through the second port. The dispenser is fluidly coupled to the common conduit through the third port.

In certain aspects, a method of delivering oxygen is provided. The method includes providing a connection box including a common conduit and first, second, and third ports fluidly coupled to the common conduit. A liquid oxygen source is fluidly coupled to the common conduit through the first port. A gaseous oxygen source is fluidly coupled to the common conduit through the second port. A dispenser is fluidly coupled to the common conduit through the third port. Oxygen is supplied from one of the liquid oxygen source and the gaseous oxygen source to the common conduit. Oxygen is then delivered from the common conduit to the dispenser. Oxygen can also be supplied from the liquid oxygen source and the gaseous oxygen source to the common conduit. For example, the oxygen from the liquid oxygen source and the gaseous oxygen source can be combined in the common conduit. The dispenser can also be fluidly coupled to the common conduit through a manifold that includes a plurality of dispensers and oxygen from the common conduit can be delivered to the plurality of dispensers.

In various aspects, a connection box for use in an oxygen delivery system is provided. The connection box includes a common conduit and first, second, and third ports fluidly coupled to the common conduit. The first port is fluidly coupled to the common conduit through first and second check valves. The second port is fluidly coupled to the common conduit through third and fourth check valves. The third port is fluidly coupled to the common conduit through a fifth check valve.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a schematic diagram of an embodiment of an oxygen delivery system according to the present technology.

FIG. 2 is a schematic diagram of another embodiment of an oxygen delivery system according to the present technology, where a connection box is depicted in a cutaway view to show further aspects of the connection box.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding the methods disclosed, the order of the steps presented is exemplary in nature, and thus, the order of the steps can be different in various embodiments. Except in the examples, or where otherwise expressly indicated, all numerical quantities in this description indicating amounts of material or conditions of reaction and/or use are to be understood as modified by the word “about” in describing the broadest scope of the technology.

The present technology provides ways to couple both a liquid oxygen source and a gaseous oxygen source to one or more dispensers for administering oxygen to one or more persons. In particular, a connection box is configured to have a common conduit for delivering the liquid oxygen source, the gaseous oxygen source, or both the liquid oxygen source and the gaseous oxygen source to the one or more dispensers. The connection box can include one or more check valves on each side of the common conduit to prevent back-flow. Multiple check valves can be positioned in the connection box to provide redundancy. The oxygen delivery system can also be referred to as a Total Oxygen Administration Device (i.e., “TOAD”).

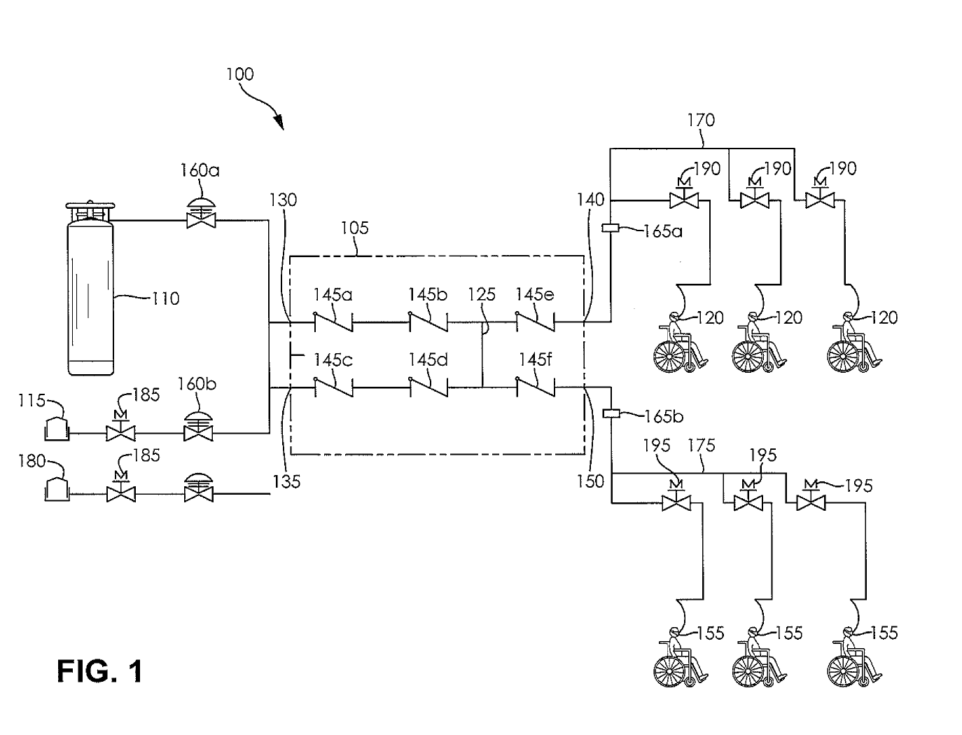

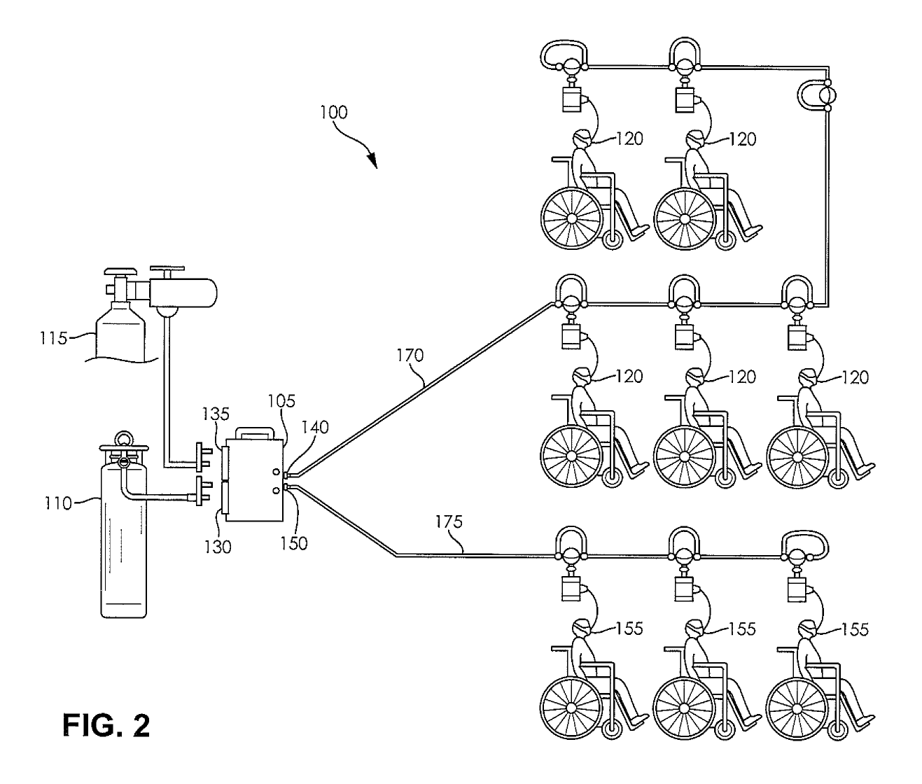

With reference to FIGS. 1 and 2, embodiments of oxygen delivery systems 100 are shown where common reference numerals indicate like features. The oxygen delivery system 100 includes a connection box 105, a liquid oxygen source 110, a gaseous oxygen source 115, and at least one dispenser 120. The connection box 105 includes a common conduit 125, a first port 130, a second port 135, and a third port 140, the ports 130, 135, 140 being fluidly coupled to the common conduit 125. The liquid oxygen source 110 is fluidly coupled to the common conduit 125 through the first port 130. The gaseous oxygen source 115 is fluidly coupled to the common conduit 125 through the second port 135. The dispenser 120 is fluidly coupled to the common conduit 125 through the third port 140.

Various check valves 145a-f can be incorporated into the system to prevent back-flow at various points. For example, at least one of the first port 130, the second port 135, and third port 140 can be fluidly coupled to the common conduit 125 through a check valve, such as check valves 145a-e. At least one of the first port 130 and the second port 135 can further be fluidly coupled to the common conduit 125 through two check valves, such as check valves 145a-b and 145c-d respectively. The check valves 145a-f used in the system 100 can include the following operating parameters: Max. Pressure: 500 PSIG; operating temp. range of −20° to +180° F.; a spring-close design to prevent slamming when the valve closes to stop backward flow; a body can be brass; at least 1 psi can be required to open the valves; can be installed in any direction; connections are NPT; can have a fluoroelastomer seal(s).

The connection box 105 can also include a fourth port 150 that is fluidly coupled to the common conduit 125 and at least one other dispenser 155 is fluidly coupled to the fourth port 150. For example, as shown in the Figures another plurality of dispensers 155 can be coupled to the fourth port 150. With particular reference to FIG. 2, the system 100 can include the following arrangement of ports 130, 134, 140, 150 and check valves 145a-f: the first port 130 is fluidly coupled to the common conduit 125 through first and second check valves 145a-b, the second port 135 is fluidly coupled to the common conduit 125 through third and fourth check valves 145c-d, the third port 140 is fluidly coupled to the common conduit 125 through a fifth check valve 145e, and the fourth port 150 is fluidly coupled to the common conduit 125 through a sixth check valve 145f.

The system 100 can also be controlled and regulated in various ways. At least one of the liquid oxygen source 110 and the gaseous oxygen source 115 can be fluidly coupled to the common conduit 125 through a control valve 160a-b. In certain embodiments, the connection box 105 can comprise the control valve 160a-b (not shown), the control valve 160a-b can be part of the liquid oxygen source 110 and/or the gaseous oxygen source 115 (not shown), or the control valve 160a-b can be located along a conduit fluidly coupling the liquid oxygen source 110 and/or the gaseous oxygen source 115 to the common conduit 125 as shown. The control valve 160a-b can be used to regulate a pressure within the common conduit 125. Moreover, the control valve 160a-b can be responsive to a pressure within the common conduit 125 and can provide a measurement of the pressure therein. Suitable examples of control valves 160a-b include medical gas pressure reducers or regulators having a 20 micron sintered metal inlet filter along with a single stage, piston type, back pressure compensated, pressure reducer cartridge having an internal relief valve, where the “click style” flowmeter module can have 12 flow settings and can operate in various positions or while moving. Commercial examples are available from Flotec, Inc. (Indianapolis, Ind.).

Fluid coupling the liquid oxygen source 110 and the gaseous oxygen source 115 to the connection box 105 can include the respective control valves 160a-b, where the control valves 160a-b can be integrally regulating; i.e., the control valve 160a-b can provide the same pressure regardless of the oxygen source. In this way, multiple conduits can be used to fluidly couple the respective components, each having a control valve, where various oxygen sources (liquid or gaseous) can be coupled thereto. Standby conduits can be fitted to the connection box to provide additional oxygen source(s) as needed. Manual valves, such as manual valves 185 shown in FIG. 2, can also be positioned in the conduit between the oxygen sources (i.e., liquid oxygen source and gaseous oxygen sources) and the control valves 165a-b. Using two or more oxygen sources allows gas pressure to be maintained in the system 100 during changeover of a depleted oxygen source.

Oxygen output to the dispensers 120, 155 can be further controlled using a flow meter 165a-b, where the respective dispenser 120, 155 is fluidly coupled to the common conduit 125 through a respective flow meter 165a-b. In various embodiments, the connection box 105 can comprise the flow meter 165a-b (not shown), the flow meter 165a-b can be part of the respective dispenser 120, 155, or the flow meter 165a-b can be located along a conduit fluidly coupling the dispenser 120, 155 to the common conduit 125 as shown. Suitable flow meters 165a-b include medical gas flowmeters, such as calibrated, 12 position, fixed orifice, non-gravity sensitive devices that provide 30 micron inlet filtration and function as back pressure compensated devices based on the “Perfect Gas Law,” that can provide flows as low as 1/50th of a liter (i.e., 20 cc) per minute up to 60 LPM. Such flow meters 165a-b are described in U.S. Pat. No. 6,026,854, which is incorporated herein by reference, and are commerically available from Flotec, Inc. (Indianapolis, Ind.).

The dispensers 120, 155 can be configured in various ways. For example, each dispenser 120, 155 can be a nasal cannula, a face mask, or other type of oxygen dispensing device. As shown in the Figures, the dispensers 120, 155 are fluidly coupled to the common conduit 125 through respective manifolds 170, 175 that each comprise a plurality of dispensers 120, 155. As shown in FIG. 1, the manifolds 170, 175 can have different numbers of dispensers 120, 155, where the manifold 170 fluidly coupled to the third port 140 has five dispensers 120 and the manifold 175 fluidly coupled to the fourth port 150 has three dispensers 155. Where the plurality of dispensers 120, 155 is fluidly coupled to the common conduit 125 through a flow meter 165a-b, the flow meter 165a-b can be configured to deliver an oxygen flow rate to the dispensers 120, 155 in proportion to the number of dispensers 120, 155 comprised by the respective manifold 170, 175. As shown in FIG. 2, the manifolds 170, 175 can comprise the flow meters 165a-b between the dispensers 120, 155 and the common conduit 125. The manifolds 170, 175 can further include manual valves 190, 195 located at or near each of the one or more dispensers 120, 155. Accordingly, an output of oxygen can be started/stopped at each dispenser 120, 155 as a person is treated by the system 100. Suitable manifolds 170, 175 include the mass casualty manifolds (Multilatorr) and assemblies commercially available from Flotec, Inc. (Indianapolis, Ind.).

Various oxygen sources can be used in the system 100. For example, one of the liquid oxygen source and the gaseous oxygen source can be a portable container so that the system can be readily deployed and transported as needed, Extra oxygen gas sources can be on hand to replace depleted sources. As shown in FIG. 2, an additional gaseous oxygen source 180 can supplement the system 100. The additional gaseous oxygen source 180 can be used to replace the original gaseous oxygen source 115 when it is depleted while the liquid oxygen source 110 maintains a constant oxygen supply to the common conduit 125 during the changeover of the gaseous oxygen sources 115, 180.

The present technology further provides various methods of delivering oxygen. In one embodiment, a method of delivering oxygen includes providing a connection box 105 including a common conduit 125 and first, second, and third ports 130, 135, 140 fluidly coupled to the common conduit 125. A liquid oxygen source 110 is fluidly coupled to the common conduit 125 through the first port 130. A gaseous oxygen source 115 is fluidly coupled to the common conduit 125 through the second port 135. And a dispenser 120 is fluidly coupled to the common conduit 125 through the third port 140. Oxygen is supplied from one of the liquid oxygen source 110 and the gaseous oxygen source 115 to the common conduit 125. The oxygen is then delivered from the common conduit 125 to the dispenser 120.

The methods of delivering oxygen can further include the following aspects. The supplying step can include supplying oxygen from the liquid oxygen source 110 and the gaseous oxygen source 115 to the common conduit 125. That is, both the liquid oxygen source 110 and the gaseous oxygen source 115 can simultaneously supply oxygen to the common conduit 125. Oxygen from the liquid oxygen source 110 and the gaseous oxygen source 115 can therefore be combined in the common conduit 125. In this way, if one of the oxygen sources becomes depleted during use, the supply of oxygen to the common conduit 125 is not interrupted. For example, the gaseous oxygen source 115 may become depleted and therefore the gaseous oxygen source 115 can be removed from the second port 135 and the additional gaseous oxygen source 180 can be coupled to the second port 135. Alternatively, the liquid oxygen source 110 may become depleted and therefore the liquid oxygen source 110 can be removed from the first port 130 and the additional gaseous oxygen source 180 can be coupled to the first port 130. In another embodiment, the depleted liquid oxygen source 110 can be replaced by another liquid oxygen source (not shown).

As described for the various oxygen delivery systems, the dispenser 120 can be fluidly coupled to the common conduit 125 through a manifold 170 that comprises a plurality of dispensers 120. Delivering oxygen from the common conduit 125 to the dispenser 125 can therefore include delivering the oxygen from the common conduit 125 to the plurality of dispensers 120. Various manifolds 170, 175 can be used, each having a particular number of dispensers 120, 155, with flow meters 165a-b adapted to deliver oxygen at a rate based on the number of dispensers 120, 155.

The present systems and methods provide ways to administer oxygen at a disaster scene or for other situations that require medical grade oxygen administration for patient care. The technology can facilitate improved medical response and treatment in such situations. In particular, gaseous oxygen sources, such as high pressure oxygen (gas) bottles can be stored a long time but typically provide a limited quantity of oxygen. These high pressure oxygen (gas) bottles may therefore require frequent change-outs while the system is in operation. Conversely, liquid oxygen sources can provide a larger quantity of oxygen per volume than gaseous oxygen sources, but liquid oxygen can effectively boil off over time, making liquid oxygen sources less suitable for long term storage. Liquid oxygen sources can include variable gas liquid (VGL) Dewar containers. Such containers can convert liquid oxygen into gaseous oxygen to feed into the system. Suitable liquid oxygen containers are commercially available from Taylor-Wharton (Theodore, Ala.).

The ability to employ both liquid oxygen sources and gaseous oxygen sources allows the present systems and methods to seamlessly switch between oxygen sources and continuously provide oxygen during replacement of a depleted oxygen source. Providing uninterrupted oxygen to injured persons can improve the time and/or effectiveness of recovery. The present system is also portable and adaptable to particular needs at a disaster site or other treatment sites. For example, one or more manifolds can be coupled to the connection box where each manifold can have one or more dispensers, where the number of dispensers can be tailored to the number of persons needing treatment.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

What is claimed is:

1. An oxygen delivery system comprising:
a connection box including a common conduit and first, second, and third ports fluidly coupled to the common conduit;
a liquid oxygen source fluidly coupled to the common conduit through the first port;
a gaseous oxygen source fluidly coupled to the common conduit through the second port; and
a dispenser fluidly coupled to the common conduit through the third port.

2. The system of claim 1, wherein one of the first port, the second port, and the third port is fluidly coupled to the common conduit through a check valve.

3. The system of claim 1, wherein one of the first port and the second port is fluidly coupled to the common conduit through two check valves.

4. The system of claim 1, wherein the connection box includes a fourth port fluidly coupled to the common conduit and another dispenser.

5. The system of claim 4, wherein the first port is fluidly coupled to the common conduit through a first check valve and a second check valve, the second port is fluidly coupled to the common conduit through a third check valve and a fourth check valve, the third port is fluidly coupled to the common conduit through a fifth check valve, and the fourth port is fluidly coupled to the common conduit through a sixth check valve.

6. The system of claim 1, wherein one of the liquid oxygen source and the gaseous oxygen source is fluidly coupled to the common conduit through a control valve.

7. The system of claim 6, wherein the connection box comprises the control valve.

8. The system of claim 1, wherein the dispenser is fluidly coupled to the common conduit through a flow meter.

9. The system of claim 8, wherein the connection box comprises the flow meter.

10. The system of claim 1, wherein one of the liquid oxygen source and the gaseous oxygen source comprises a portable container.

11. The system of claim 1, wherein the dispenser comprises one of a nasal cannula and a face mask.

12. The system of claim 1, wherein the dispenser is fluidly coupled to the common conduit through a manifold comprising a plurality of dispensers.

13. The system of claim 12, wherein the plurality of dispensers is fluidly coupled to the common conduit through a flow meter, the flow meter configured to deliver an oxygen flow rate to the dispensers in proportion to a number of the dispensers comprised by the manifold.

14. The system of claim 13, wherein the connection box comprises the flow meter.

15. The system of claim 13, wherein the manifold comprises the flow meter.

16. A method of delivering oxygen comprising:
providing a connection box including a common conduit and a first port, a second port, and a third port fluidly coupled to the common conduit; a liquid oxygen source fluidly coupled to the common conduit through the first port; a gaseous oxygen source fluidly coupled to the common conduit through the second port; and a dispenser fluidly coupled to the common conduit through the third port;
supplying oxygen from at least one of the liquid oxygen source and the gaseous oxygen source to the common conduit; and
delivering the oxygen from the common conduit to the dispenser.

17. The method of claim 16, wherein the supplying step comprises supplying oxygen from both the liquid oxygen source and the gaseous oxygen source to the common conduit.

18. The method of claim 16, wherein the supplying step comprises combining oxygen from the liquid oxygen source and the gaseous oxygen source in the common conduit.

19. The method of claim 16, wherein the dispenser is fluidly coupled to the common conduit through a manifold comprising a plurality of dispensers and the delivering step comprises supplying the oxygen from the common conduit to the plurality of dispensers.

20. A connection box for use in an oxygen delivery system comprising a common conduit and a first port, a second port, and a third port fluidly coupled to the common conduit; wherein the first port is fluidly coupled to the common conduit through a first check valve and a second check valve, the second port is fluidly coupled to the common conduit through a third check valve and a fourth check valve, and the third port is fluidly coupled to the common conduit through a fifth check valve.
